(19) United States
(12) Patent Application Publication
(10) Pub. No.: US 2005/0198704 A1
(43) Pub. Date: Sep. 8, 2005

(54) Chloroplast transgenesis of monocots: bioconfined genetically engineered monocot crops that will eliminate transgene flow

(75) Inventor: Masomeh B. Sticklen, East Lansing, MI (US)

Correspondence Address: Ian C. McLeod;McLEOD & MOYNE, P.C., 2190 Commons Parkway, Okemos, MI, 48864, US

(73) Assignee: Board of Trustees of Michigan State University, East Lansing, MI (US)

(21) Appl. No.: 11/100,270

(22) Filed: Apr. 6, 2005

Related U.S. Application Data
(63) Continuation-in-part of application No. 09/981,900, filed on Oct. 18, 2001.
(60) Provisional application No. 60/561,476, filed on Apr. 12, 2004.
(60) Provisional application No. 60/242,408, filed on Oct. 20, 2000.

(51) Int. Cl. A01H 1/00; C12N 15/82

(52) U.S. Cl. 800/278; 800/282

(57) ABSTRACT

The present invention discloses transgenic monocot plants in which the plastid genome has been genetically engineered. The bioconfined genetically engineered monocot crops have transgene-free pollen grains which eliminate or dramatically reduce transgene flow. The present invention discloses plastid transgenesis technology having the additional advantages of the absence of gene silencing and position effect variation, the ability to express polycistronic messages from a single promoter, integration via a homologous recombination process that facilitates targeted gene replacement and precise transgene control, and sequestration of foreign proteins in the organelle which prevents adverse interaction with the cytoplasmic environment.

### CROSS-REFERENCE TO RELATED APPLICATIONS

This application claims priority to provisional Patent Application Ser. No. 60/561,476 filed Apr. 12, 2004. This application is a continuation in part of U.S. application Ser. No. 09/981,900, filed Oct. 18, 2001, which claims priority to Provisional Application 60/242,408, filed Oct. 20, 2000.

### STATEMENT REGARDING FEDERALLY SPONSORED RESEARCH OR DEVELOPMENT

Not Applicable

### BACKGROUND OF THE INVENTION

(1) Field of the Invention

The present invention relates to transgenic monocot plants. In particular, the invention relates to monocot plants in which the plastid genome has been genetically engineered. The bioconfined genetically engineered monocot crops have transgene-free pollen grains which eliminate or dramatically reduce transgene flow.

(2) Description of the Related Art

Genetic engineering of corn was initially developed to control biotic and abiotic stresses. Soon after its development, it was realized that low value abundant foliage such as lignocellulosic biomass or stover of this major U.S. crop could be engineered with microbial genes and then used for production of biofuel energy and bio-based products including industrial enzymes, pharmaceuticals and polymers. Ethanol produced from biomass is currently the subject of extensive research, development and demonstration efforts. However, most efforts have been made on non-transgenic biomass, and very little effort has been expanded toward producing the needed cellulase enzymes as part of transgenic plants. As is well know, the United States transportation sector is 99% dependent on petroleum, approximately 60% of which is imported. Our country, and much of the developed world, is highly vulnerable to petroleum supply disruptions. Ethanol is an excellent fuel and, with current supply levels (mostly based on corn grain) also a valuable gasoline extender/octane enhancer. However, corn grain-based ethanol can never provide us the volumes of fuel needed to seriously impact our liquid fuel supplies.

Insertion of genes encoding for enzymes can improve the availability of fermentable sugars for ethanol production from corn biomass, i.e., the leaves and stalks. Corn biomass is rarely used for ethanol production because of the cost in degrading the leaves and stalks comprising lignins and cellulose, generally in the form of lignocellulose, to fermentable sugars. The lignocellulose in the stalks and leaves of corn biomass represents a tremendous source of untapped energy that goes unused because of the difficulty and cost of converting it to fermentable sugars.

Currently, there are four technologies available to convert cellulose to fermentable sugars. These are concentrated acid hydrolysis, dilute acid hydrolysis, biomass gasification and fermentation, and enzymatic hydrolysis. Concentrated acid hydrolysis is based on concentrated acid de-crystallization of cellulose followed by dilute acid hydrolysis to sugars at near theoretical yields. Separation of acid from sugars, acid recovery, and acid re-concentration are critical unit operations. The concentrated sulfuric acid process has been commercialized in the past, particularly in the former Soviet Union, Germany, and Japan. However, these processes were only successful during times of national crisis, when economic competitiveness of ethanol production could be ignored.

Dilute acid hydrolysis occurs in two stages to maximize sugar yields from the hemicellulose and cellulose fractions of biomass. The first stage is operated under milder conditions to hydrolyze hemicellulose, while the second stage is optimized to hydrolyze the more resistant cellulose fraction. Liquid hydrolyzates are recovered from each stage, neutralized, and fermented to ethanol. As indicated earlier, Germany, Japan, and Russia have operated dilute acid hydrolysis percolation plants off and on over the past 50 years. However, the technology remains non-competitive for the conversion of cellulose to fermentable sugars for production of ethanol.

In biomass gasification and fermentation, biomass is converted to a synthesis gas, which consists primarily of carbon monoxide, carbon dioxide, and hydrogen) via a high temperature gasification process. Anaerobic bacteria are then used to convert the synthesis gas into ethanol.

In early processes embracing enzymatic hydrolysis of biomass to ethanol, the acid hydrolysis step was replaced with an enzyme hydrolysis step. This process scheme was often referred to as separate hydrolysis and fermentation (SHF) (Wilke et al, Biotechnol. Bioengin. 6: 155-175 (1976)). In SHF, pretreatment of the biomass is required to make the cellulose more accessible to the enzymes. Many pretreatment options have been considered, including both thermal and chemical steps. The most important process improvement made for the enzymatic hydrolysis of biomass was the introduction of simultaneous saccharification and fermentation (SSF) U.S. Pat. No. 3,990,944 to Gauss et al. and U.S. Pat. No. 3,990,945 to Huff et al.). This process scheme reduced the number of reactors involved by eliminating the separate hydrolysis reactor and, more importantly, avoiding the problem of product inhibition associated with enzymes.

In the presence of glucose, β-glucosidase stops hydrolyzing cellobiose. The build up of cellobiose, in turn, shuts down cellulose degradation. In the SSF process scheme, cellulase enzyme and fermenting microbes are combined. As sugars are produced by the enzymes, the fermentative organisms convert them to ethanol. The SSF process has, more recently, been improved to include the co-fermentation of multiple sugar substrates in a process known as simultaneous saccharification and co-fermentation (SSCF) (www.ott.doe.gov/biofuels/enzymatic.html).

While cellulase enzymes are already commercially available for a variety of applications. Most of these applications do not involve extensive hydrolysis of cellulose. For example, the textile industry applications for cellulases require less than 1% hydrolysis. Ethanol production, by contrast, requires nearly complete hydrolysis. In addition, most of the commercial applications for cellulase enzymes represent higher value markets than the fuel market. For these reasons, enzymatic hydrolysis of biomass to ethanol remains non-competitive.

However, while the above processes have focused on converting cellulose to fermentable sugars or other products, much of the cellulose in plant biomass is in the form of lignocellulose. Lignin is a complex macromolecule consisting of aromatic units with several types of inter-unit linkages. In the plant, the lignin physically protects the cellulose polysaccharides in complexes called lignocellulose. To degrade the cellulose in the lignocellulose complexes, the lignin must first be degraded. While lignin can be removed in chemi-mechanical processes that free the cellulose for subsequent conversion to fermentable sugars, the chemi-mechanical processes are inefficient. Ligninase and cellulase enzymes, which are produced by various microorganisms, have been used to convert the lignins and cellulose, respectively, in plant biomass to fermentable sugars. However, the cost for these enzymes is expensive, about six dollars a pound. As long as the cost to degrade plant biomass remains expensive, the energy locked up in the plant biomass will largely remain unused.

An attractive means for reducing the cost of degrading plant biomass is to make transgenic plants that contain cellulases. For example, WO 98/11235 to Lebel et al. discloses transgenic plants that express cellulases in the chloroplasts of the transgenic plants or transgenic plants wherein the cellulases are targeted to the chloroplasts. Preferably, the cellulases are operably linked to a chemically-inducible promoter to restrict expression of the cellulase to an appropriate time. However, because a substantial portion of the cellulose in plants is in the form of lignocellulose, extracts from the transgenic plants are inefficient at degrading the cellulose in the lignocellulose.

U.S. Pat. No. 5,981,835 to Austin-Phillips et al. discloses transgenic tobacco and alfalfa which express the cellulases E2, or E3 from Thermomononospora fusca. The genes encoding the E2 or E3, which were modified to remove their leader sequence, were placed under the control of a constitutive promoter and stably integrated into the plant genome. Because the leader sequence had been removed, the E2 or E3 product preferentially accumulated in the cytoplasm of the transgenic plants. However, because the cellulase can leak out of the cytoplasm and into the cell wall where it can degrade cellulose in the cell wall, the growth of the transgenic plants can be impaired.

While the above transgenic plants are an improvement, accumulation of cellulytic enzymes in the cytoplasm of a plant is undesirable since there is the risk that the cellulase can leak out from the cytoplasm and injure the plant. For example, research has shown that plants such as the avocado, bean, pepper, peach, poplar, and orange also contain cellulase genes, which are activated by ethylene during ripening and leaf and fruit abscission. Therefore, transgenic plants which contain large quantities of cellulase in the cytoplasm are particularly prone to damage. Furthermore, the cellulases accumulate in all tissues of the plant which can be undesirable. Restriction of cellulase expression to plastids is desirable because it reduces the risk of plant damage due the cellulases leaking from the cell. However, for most crop plants, it has been difficult to develop a satisfactory method for introducing heterologous genes into the genome of plastids.

For production of ligninases to use in degrading lignins, the ligninases of choice are from the white-rot fungus Phanerochaete chrysosporium. One of the major lignin-degrading, extracellular enzymes produced by P. chrysosporium is lignin peroxidase (LIP). Potential applications of LIP include not only lignin degradation but also biopulping of wood and biodegradation of toxic environmental pollutants. To produce large quantities of LIP, the fungus can be grown in large reactors and the enzyme isolated from the extracellular fluids. However, the yields have been low and the process has not been cost-effective. Production of recombinant LIP in E. coli, in the fungus Trichoderma reesei, and baculovirus have been largely unsuccessful. Heterologous expression of lignin-degrading manganese peroxidase in alfalfa plants has been reported; however, the transgenic plants had reduced growth and expression of the enzyme was poor (Austin et al, Euphytica 85: 381-393 (1995)).

Although difficult to sufficiently and cheaply produce ligninases in non-plant systems, ligninases have evoked worldwide interest because of their potential in degrading a variety of toxic xenobiotic compounds such as PCBs and benzo(a)pyrenes in the environment (Yadav et al, Appl. Environ. Microbiol. 61: 2560-2565 (1995); Reddy, Curr. Opin. Biotechnol. 6: 320-328 (1995); Yadav et al, Appl. Environ. Microbiol. 61: 677-680 (1994)).

The major concern with the commercial application of genetically engineered crops is the unintended transfer of transgenes via pollen flow to nearby cross-breedable species (Riegel, M. A., Lamond, M., Preston, C., Powles, S. B., and Roush, R. T. (2002). Pollen-mediated movement of herbicide resistance between commercial canola fields. Science 296, 2386-2388). For example, recently in Iowa, a local producer has been required to destroy 155 acres of corn because it could have been cross-pollinated with ProdiGene's engineered corn producing a biopharmaceutical (Fox, J. L. (2003). Puzzling industry response to ProdiGene fiasco. Nat. Biotechnol. 21, 3-4.). European countries have banned the transfer of transgenic maize and any maize that may be contaminated with transgenes, to Europe. Economically, this has become a large loss for the U.S. maize producers, maize biotechnology companies and overall for the U.S. economy. Incidents such as STARLINK® corn (Aventis, Strasbourg, France) being accidentally mixed with human corn seeds, and the report of the death of Monarch butterflies which were fed Bt-corn pollens are two simple examples of events which have economic repercussions impacting maize nuclear transgenesis technology in our own country.

One approach to address the concern about transgene flow into the environment is to confine the transgenes within the plant in such a manner that they will not enter the genetic content of pollen grains. The genetic content of the chloroplast in maize and other monocots are maternally inherited. This characteristic of the plastid genome makes chloroplast transgenesis an ideal technology for creating transgenic corn, since the transgenes will not be carried by pollen to any plants which are capable of being cross-fertilized. To date, there have been no reports on the successful transfer of any gene to maize plastids, nor any transfer of any cellulase gene to the corn nuclear genome. There are reports on expression of transgenes in chloroplasts of tobacco, Arabidopsis, potato, tomato, Brassica napus, and Lesquerella fendleri through Biolistic® bombardment of leaf tissue explants, and rice through Biolistic® bombardment of cell suspensions. Also there are reports that address the stable expression of gus gene in chloroplasts of tobacco through polyethylene glycol-mediated transformation of leaf protoplasts (Golds T., Maliga, P. and Koop, H. (1993). Stable plastid transformation in PEG-treated protoplasts of Nicotiana tobaccum. Biotechnology. 11: 95-97.).

U.S. Pat. No. 6,013,860 to Himmel et al. discloses transgenic plants which express the cellulase E1 from Acidothermus cellulolyticus. The gene encoding E1, which was modified to remove the leader region, was placed under the control of a plastid specific promoter and preferably integrated into the plastid genome. Since the leader sequence had been removed, the E1 product accumulated in the plastid.

U.S. Pat. No. 5,767,368 to Zhong et al. describes a very powerful system of multimeristem primordial regeneration for maize (Zhong H., Srinivasan, C. and Sticklen, M. (1992a). Morphogenesis of corn (Zea mays L.) in vitro II. Transdifferentiation of shoots, tassels, and ear primordial from corn shoot tips. Planta 187: 483-489.; Zhong et al., 1992b), oat (Zhong, H., Warkentin, D., Sun, B., Zhang, S., Wu, T., Wu, R., and Sticklen, M. (1996a). Analysis of the functional activity of the 1.4 kb 5′-region of the rice actin 1 gene in stable transgenic plants of maize (Zea mays L.). Plant Science 116:73-84.), wheat (Ahmad A., Zhong, H., Wang, W. and Sticklen, M. (2002). Shoot apical meristem: In vitro regeneration and morphogenesis in wheat (Triticum aestivum L.). In-Vitro Cellular & Developmental Biology (IVCDB)-PLANT. 38: 163-167), millet (Devi, P. and Sticklen, M. B. (2002). Genetic engineering of pearl millet through meristem primordia system. Euphytica (125): 45-50), and sorghum (Zhong H., Wang, W. and Sticklen, M. (1998). In vitro morphogenesis of Sorghum bicolor (L.) Moench: Efficient plant regeneration from shoot apices. J. Plant Physiol. 153: 719-726) and this system can be used to genetically engineer nuclear genomes of maize (Zhong, H., Warkentin, D., Sun, B., Zhang, S., Wu, T., Wu, R., and Sticklen, M. (1996a). Analysis of the functional activity of the 1.4 kb 5′-region of the rice actin 1 gene in stable transgenic plants of maize (Zea mays L.). Plant Science 116:73-84; Zhong H. and M. Sticklen (1996b). Production of multiple shoots from shoot apical meristems of oat (Avena sativa L.). J. Plant Physiol. 148:667-671; Zhong H., Teymouri, F., Chapman, B., Maqbool, S., Sabzikar, R., El-Maghraby, Y., Dale, B. and Sticklen, M. (2003). The dicot pea (Posum sativum L.) rbcS transit peptide directs the Alcaligenes eutrophus polyhydroxybutyrate enzymes into the monocot maize (Zea mays L.) chloroplasts. J. Plant Science. Vol. 1-8. In press), oat (Maqbool S., Zhong, H., El-Maghraby, Y., Wang, W., Ahmad, A., Chai, B. and Sticklen, M. (2002). Competence of oat (Avena sativa L.) shoot apical meristems for integrative transformation, inherited expression and osmatic tolerance of hva1 transgene. Theor. Appli. Genet. 105:201-208) and millet (Devi, P. and Sticklen, M. B. (2002). Genetic engineering of pearl millet through meristem primordia system. Euphytica (125): 45-50). Tissue-specific transgene expression and chloroplast targeting of products (heterologous proteins) of three microbial polyhydroxybutyrate (phb) genes (Zhong H., Teymouri, F., Chapman, B., Maqbool, S., Sabzikar, R., El-Maghraby, Y., Dale, B. and Sticklen, M. (2003). The dicot pea (Posum sativum L.) rbcS transit peptide directs the Alcaligenes eutrophus polyhydroxybutyrate enzymes into the monocot maize (Zea mays L.) chloroplasts. J. Plant Science. Vol. 1-8. In press) have successfully limited the production of genes products to green parts of maize (i.e. foliage).

While the related art teach plastid transformation of tobacco and various other plants, a need remains for an economical method for making transgenic monocot plants with genes incorporated into the plastid genome of the monocot plant wherein the genes and protein expression is biocompartmentalized so that the enzymes can accumulate without damaging the transgenic plant and the transgenes can be kept out of the pollen, so that surrounding wild monocot plants which are cross-breedable will not be contaminated.

### OBJECTS

Therefore, it is an object of the present invention to provide transgenic monocot plants which incorporate the transgenes within the plant plastids.

It is further an object of the present invention to provide a method of making the transgenic monocot plants which incorporate the transgenes within the plant plastids.

It is still further an object of the present invention to provide transgenic monocot plants which degrade lignocellulose to fermentable sugars.

These and other objects will become increasingly apparent by reference to the following description.

### SUMMARY OF THE INVENTION

The present invention provides a method of genetically engineering a monocot plant so that one or more transgenes stably integrate within plastid genomic DNA of the monocot plant comprising: providing a monocot plant and a targeting construct comprising one or more transgenic sequences; introducing the targeting DNA sequence into the monocot plant by means of transformation of multimeristems; selecting transformed monocot plant cells; and growing the transformed monocot plant, so that the transformed monocot plant has one or more transgenes stably integrated within the plastid genomic DNA.

In further embodiments of the method the targeting construct further comprises a first targeting sequence complimentary to a first plastid genomic DNA sequence of a monocot plastid, a second targeting sequence complimentary to a second plastid genomic DNA sequence of a monocot plastid, a plastid specific promoter linked between the first targeting sequence and the second targeting sequence, wherein the one or more transgenic DNA sequences are between the first targeting sequence and the second targeting sequence and are operationally linked to the plastid specific promoter segment. In still further embodiments of the method the transformed monocot plant cells are selected by means of herbicide resistance or antibiotic resistance. In still further embodiments of the method the one or more transgenic DNA sequences encode for at least one enzyme. In still further embodiments of the method the monocot plant is maize. In still further embodiments of the method the plastid is a chloroplast.

In further embodiments of the method the one or more transgenic DNA sequences confer herbicide resistance, insect resistance, or environmental stress resistance. In still further embodiments of the method the targeting construct further comprises a DNA encoding a selectable marker operably linked to a constitutive promoter. In still further embodiments of the method the DNA encoding the selectable marker provides the transgenic plant with resistance to an antibiotic, an herbicide, or to environmental stress. In still further embodiments of the method the DNA encoding resistance to the herbicide is a DNA encoding phosphinothricin acetyl transferase which confers resistance to the herbicide phosphinothricin. In still further embodiments of the method the DNA encoding the selectable marker provides the transgenic plant with resistance to an antibiotic selected from the group consisting of hygromycin, streptomycin, spectinomycin, G418, and paromomycin. In still further embodiments of the method the plastid specific promoter is selected from the group consisting of Prrn, PrpoB, PrbcL, PpsbA and PpsbD.

### BRIEF DESCRIPTION OF THE DRAWINGS

FIG. 1 illustrates a map of a chloroplast-specific plasmid construct for maize. The illustrated region is flanked by trnv-rps 12/7 plastid derived sequences. aadA represents the gene encoding aminoglycoside adenyl transferase, which renders resistance to spectinomycin and streptomycin. gfp is the green fluorescent protein gene from jellyfish. TpsbA represents the 3′ untranslated region of the psbA gene. PpsbA represents the regulatory region of the psbA gene. Prrn represents the regulatory region of the prrn promoter. Trps16 represents the 3′ region of the ribosomal protein gene. BglI, MseI, EcoRV, NcoI, BamHI, ClaI and XbaI identify the corresponding restriction endonuclease recognition sites. Insert sizes are designated in kilobases (kB).

### DETAILED DESCRIPTION OF THE INVENTION

All patents, patent applications, government publications, government regulations, and literature references cited in this specification are hereby incorporated herein by reference in their entirety. In case of conflict, the present description, including definitions, will control.

The term “monocot” as used herein refers to all monocotyledonae plants including, but not limited to cereal plants such as maize, wheat, barley, oats, rye, rice, buckwheat, millet, and sorghum. Additionally monocot plants include switchgrass, and other perennial grasses. Other monocots include such plants as sugar cane.

The term “plastid” as used herein includes, but is not limited to amyloplasts, chloroplasts, chromoplasts, elaioplasts, etioplasts and leucoplasts.

The term “homologous recombination” as used herein refers to the recombination of two identical or similar DNA strands within a cell.

The term “cellulase” as used herein is a generic term that includes endoglucanases such as the EI beta-1,4-endoglucanase precursor gene (e1) of Acidothermus cellulolyticus and exoglucanases such as the cellobiohydrolase gene (cbh1) of Trichoderma reesei (also classified by some as Trichoderma longibrachiatum), the dextranase gene of Streptococcus salivarius encoding the 1,6-alpha-glucanhydrolase gene, and the beta-glucosidase gene from Actinomyces naeslundi. Endoglucanases randomly cleave cellulose chains into smaller units. Exoglucanases include cellobiohydrolases, which liberate glucose dimers (cellobiose) from the ends of cellulose chains; glucanhydrolases, which liberate glucose monomers from the ends of cellulose chains; and, beta-glucosidases, which liberate D-glucose from cellobiose dimers and soluble cellodextrins. When all four of the above enzymes are combined, they work synergistically to rapidly decrystallize and hydrolyze cellulose to fermentable sugars.

The term “lignin” as used herein is a generic term that includes both lignins and lignocelluloses.

The term “ligninase” as used herein is a generic term that includes all varieties of enzymes which degrade lignins such as the lignin peroxidase gene of Phanerochaete chrysosporium.

The term “e1” as used herein refers to the cDNA isolated from Acidothermus cellulolyticus which encodes the cellulase EI beta-1,4-endoglucanase precursor. The nucleotide sequence for the gene encoding e1 is set forth in SEQ ID NO: 1.

The term “cbh1” as used herein refers to the cDNA isolated from Trichoderma reesei that encodes the cellulase cellobiohydrolase. The nucleotide sequence for the gene encoding cbh1 is set forth in SEQ ID NO: 2.

The term “bar” as used herein refers to the phosphinothricin acetyl transferase gene (Thompson et al, EMBO J. 6: 2519-2523 (1987)). The bar gene is a selectable marker for herbicide resistance.

The present invention avoids unintentional transfer of transgenes to non-transgenic and/or crop-related wild species. Also, the normal level of gene protein product, i.e. heterologous protein, in nuclear engineered plants is 0.1 to 3% total soluble protein. The present invention increases the level of heterologous protein by several fold as compared with heterologous proteins produced via nuclear transgenesis in plants. Transgenic maize with pollens that are free from any transgenes and transgene products will improve maize transformation technology and will broaden its commercial applications. Transgenic maize commercial use will substantially improve should the issue of the U.S. transgenic maize trade with Europe be resolved, and should consumers accept the use of transgenic maize as feed, food and for it's heterologous products.

Although no technology is absolutely free from drawbacks, the powerful plastid transgenesis technology (Bock, R. (2001). Transgenic plastids in basic research and plant biotechnology. J. Mol. Biol. 312, 425-438.; Heifetz, P. B. (2000). Genetic engineering of the chloroplast. Biochimie 82, 655-666; Maliga, P. (2002). Engineering the plastid genome of higher plants. Curr. Opin. Plant Biol. 5, 164-172.; Staub, J. M. (2002). Expression of recombinant proteins via the plastid genome. In Handbook of industrial cell culture: mammalian, microbial and plant cells. S. R. Parekh and V. A. Vinci, Eds (Totowa, N.J.: Humana Press Inc.), pp. 261-280) has been identified as one of the most viable approaches to eliminate or severely reduce the transfer to transgenes through pollen grains. The fact that the chloroplast genes in most flowering plants, and specifically in maize (Moneger F., Mandaran, P., Niogret, M., Feryssinet, G. and Macher, R. (1992). Expression of chloroplast and mitochondrial genes during microsporogenesis in maize. Plant Physiol. 99 (2): 396-400) are maternally inherited, makes the chloroplast transgenesis an ideal technology for creating transgenic maize because it could accomplish the goal of pollen gene flow bioconfinement. However, to date, other than a poster abstract (Assem, S., Dhingra, A. and Danniel, H. (2002). Genetic engineering of the maize chloroplast genome. Poster Abstract Presented at the American Society of Plant Biologists meeting) that was the result of incomplete work because the research scientist left the organization, there is no published report on successful transfer of any gene to maize plastids. There are reports on expression of transgenes in chloroplasts of tobacco (Svab, Z., and Maliga, P. (1993). High-frequency plastid transformation in tobacco by selection for a chimeric aadA gene. Proc. Natl. Acad. Sci. USA. 90, 913-917; Lutz, K. A., Knapp, J. E., and Maliga, P. (2001). Expression of bar in the plastid genome confers herbicide resistance. Plant Physiol. 125, 1585-1590, Ye, G., Hajdukiewicz, P., Broyles, D., Rodriguez, D., Xu, C., Nehra, N. and Staub, J. (2001). Plastid expressed 5-endolpyruvylshikimate-3-phosphate synthase genes provide high level glyphosate tolerance in tobacco. Plant J. 25(3): 261-270), Arabidopsis (Sikdar, S. R., Serino, G., Chaudhuri, S., and Maliga, P. (1998). Plastid transformation in Arabidopsis thaliana. Plant Cell Rep. 18, 20-24), potato (Sidorov, V., Kasten, D., Pang, S., Hajdukiewicz, P., Staub, J. and Nehra, N. (1999). Stable chloroplast transformation in potato: use of green fluorescent protein as a plasmid marker. Plant J. 19(2): 209-216), tomato (Ruf S., Hermann, M., Berger, I., Carrer, H. and Bock, R. (2001). Stable genetic transformation of tomato fruit plasmids and expression of a foreign protein in fruit. Nature Biotechnology. 19(9): 870-875) Brassica napus (Hou, B. K., Zhou, Y. H., Wan, L. H., Zhang, Z. L., Shen, G. F., Chen, Z. H., and Hu, Z. M. (2003). Chloroplast transformation in oilseed rape. Transgenic Res 12, 111-114) and Lesquerella fendleri (Skarjinskaia, M., Svab, Z., and Maliga, P. (2003). Plastid transformation in Lesquerella fendleri, an oilseed Brassicacea. Transgenic Res 12, 115-122) through Biolistic® bombardment of leaf tissue explants, and progress towards transfer of genes in rice through Biolistic® bombardment of cell suspensions. Also there are reports that address the stable expression of gus gene in chloroplasts of tobacco through polyethylene glycol-mediated transformation of leaf protoplasts (Golds T., Maliga, P. and Koop, H. (1993). Stable plastid transformation in PEG-treated protoplasts of Nicotiana tobaccum. Biotechnology. 11: 95-97).

In addition to its bioconfinement, plastid transgenesis technology has other advantages, including the absence of gene silencing and position effect variation, the ability to express polycistronic messages from a single promoter, integration via a homologous recombination process that facilitates targeted gene replacement and precise transgene control, and sequestration of foreign proteins in the organelle which prevents adverse interaction with the cytoplasmic environment (Heifetz, P. B. (2000). Genetic engineering of the chloroplast. Biochimie 82, 655-666).

A variety of fungi and bacteria produce ligninase and cellulase enzymes, and based on evolutionary pressures, these fungi are able to degrade lignin or cellulose and hemicellulose of plant residues in the soil. In the laboratory, cellulases have been used to hydrolyze or convert cellulose and hemicellulose into mixtures of simple sugars that can be used in fermentation to produce a wide variety of useful chemical and fuel products, including but not limited to, ethanol, lactic acid, and 1,3-propanediol, which is an important molecular building block in the production of environmentally-friendly plastics.

The biodegradation of lignin, which comprises 20-30% of the dry mass of woody plants, is of great economic importance because this process is believed to be an important rate-limiting step in the earth's carbon cycle. Furthermore, there is considerable potential for the transformation of lignin into aromatic chemical feedstock. Also, delignification of lignocellulosic feeds has been shown to increase their digestibility by cattle by about 30%, therefore, contributing to enhanced cost effectiveness for producing milk and meat. Moreover, research on lignin biodegradation has important implications in biopulping and biobleaching in the paper industry.

The present invention provides transgenic monocot plants which produce ligninases, cellulases, or both in the leaves and straw/stalks of the plant. While the transgenic plant can be any plant which is practical for commercial production, it is preferable that the transgenic monocot plants be constructed from plants which are produced in large quantities and which after processing produce a substantial amount of leaves and stalks as a byproduct. Therefore, it is desirable that the transgenic monocot plant be constructed from cereal plants including, but not limited to, maize, wheat, barley, oats, rye, rice, buckwheat, millet, and sorghum. Other desirable plants which can be used include switchgrass, and other perennial grasses. Additionally, other monocots including but not limited to sugar cane can be used. Most preferably, the transgenic plant is constructed from monocots including maize, sugar cane, switchgrass, and other perennial grasses.

Sugar cane is a preferred plant since it is a major crop. Sugar cane fields are currently burned in locations such as Hawaii before the stems are harvested for sugar extraction. The transgenic leaves plus the stem containing the sugar can be utilized by means of the present invention for production of biofuels and other valuable chemicals.

Maize is a preferred plant because it is a major crop in the United States; approximately 60 million acres of maize are produced per year. Further, there is already a large industry built around the processing of maize grain to industrial products, which includes the production of over 1.2 billion gallons of fuel ethanol per year. Thus, fermentable sugars produced by the hydrolysis of maize stalks and leaves according to the present invention can be utilized within the large existing maize refining infrastructure. Leaves and stalks from transgenic maize made according to the present invention can be made available to this refining infrastructure in large quantities, about tens of millions of tons annually, at a current cost of about 30 dollars per ton. This cost is about one quarter of the cost of maize grain which further enhances the value of the present invention for the economical production of a wide variety of industrial products from the residue of transgenic monocot plants made according to the present invention. Furthermore, maize is preferred because it is a C-4 monocot that has very large chloroplasts. The large chloroplasts enables the chloroplasts of the transgenic maize of the present invention to accumulate higher levels of ligninases and cellulases than could be accumulated in the chloroplasts of other transgenic plants, e.g., C-3 dicots and monocots. Therefore, transgenic maize of the present invention is a particularly useful source of ligninases and cellulases.

Maize chloroplast genetic transformation has the advantages of bioconfinement of pollen gene flow, very high level of transgene expression that is important for production of therapeutic and prophylactic, and biologically based industrial compounds in plants. Working with the plastid genome has the additional advantage of eliminating the necessity to be concerned with the phenomena behind gene silencing.

Maize has been mainly transformed through the bombardment of its embryo-derived cells, which contain very small plastids. Although not impossible, this makes the chloroplast transgenesis technology less efficient than if fully developed chloroplasts of totipotent green tissues were bombarded because of the much larger size of the plastids in green cells. A system of shoot apical multimeristem bombardment, reproduction, and propagation have previously been developed for maize nuclear transgenesis as described in U.S. Pat. Nos. 5,281,529; 5,320,961; 5,767,368 to Zhong et al., hereby incorporated herein by reference. Herein, an efficient maize chloroplast transgenesis system is disclosed using maize multimeristem bombardment and a chloroplast-specific plasmid vector which leads to progenies useful for the elimination of the risks associated with prior art systems. Model plant plastid transformation has also been used to characterize the plastid transcription machinery, to understand RNA editing, the rules of mRNA translation, production of recombinant proteins in plastids and explore biotechnological applications of plastid transformation as described in U.S. patent application Nos. 2003/0200568 A1 and 2003/0088081 to Maliga et al.; U.S. Pat. Nos. 6,624,296; 6,472,586; 6,388,168; 6,376,744; 6,297,054; 5,877,402 and 5,451,513 to Maliga et al.; and U.S. Pat. No. 5,545,818 to McBride et al., all of which are hereby incorporated herein by reference.

The transgenic monocot plants of the present invention provide a plentiful, inexpensive source of fungal or bacterial ligninases and cellulases which can be used to degrade lignins and cellulose in plants to fermentable sugars for production of ethanol or for other uses which require ligninases and cellulases such as pre-treating silage to increase the energy value of lignocellulosic feeds for cows and other ruminant animals, pre-treating lignocellulosic biomass for fermentative conversion to fuels and industrial chemicals, and biodegradation of chloroaromatic environmental pollutants. Because the transgenic monocot plants of the present invention produce the ligninases, cellulases, or both therein, the external addition of ligninases and cellulases for degradation of the plant material is no longer necessary. Therefore, the present invention enables the plant biomass, which is destined to become ethanol or other products, to serve as the source of ligninase and cellulase. Furthermore, the plant material from the transgenic monocot plants of the present invention can be mixed with non-transgenic plant material. The ligninases, cellulases, or both produced by the transgenic monocot plants will degrade the lignin and cellulose of all the plant material, including the non-transgenic plant material. Thus, ligninase and cellulase degradation of plant material can be carried out more economically. When the cell walls of plants are broken down via transgenes (cellulases and ligninases), it is easier to extract other chemicals from cells for industrial purposes, whether naturally produced chemicals in cells such as succinic acid, or proteins and polymers that are produced in the plant cells via genetic engineering. Plant residues break down in the soil much faster. If fed to livestock, they get digested faster with less energy usage.

The present invention provides monocot plants wherein the stover (leaves and stems) can be used for ethanol production while avoiding unintended transfer of transgenes into any cross breedable plants that normally occur via pollen flow. However, any other transgenes can be inserted for other purposes, for example Bt genes for insect resistance, herbicide resistance genes or biopharmaceutical genes.

The present invention provides a system for chloroplast transgenesis in monocots. The monocot chloroplast transgenesis system development technology has a very high application potential to industries. Using the present invention, industries could transfer biotic and abiotic resistance, bioenergy, health-related as well as other industrial material-related genes into monocot chloroplasts for commercialization. Several private sectors are heavily engaged with production of valuable health and/or bioenergy-related compounds in maize. The present invention allows a very high level of heterologous products in plants. This too will improve the maize market.

The present invention establishes a system for the production of a system to genetically engineer monocot plastids rather than monocot nuclear genome. This system that avoids the presence of transgene(s) in pollen cells will eliminate the concerns of unintended cross-pollinations in the field. Heterologous gene transfer to corn can be utilized to make the corn more resistant to herbicides, insects, or other environmental stresses. The corn can also be used to synthesize biological products such as pharmaceutical agents. The present invention can be used for the production of biofuel ethanol and intermediate products by converting the carbohydrate portion of cellulosic biomass into sugar using transgenic monocot expressing cellulase enzymes.

To produce the present invention a series of plastid transformation vectors containing selectable markers are constructed. The cellulase genes are then transferred into chloroplasts of corn and other monocots including cereal crops via two methods. The first method includes Biolistic® bombardment of green tissues, more specifically bombardment of multimeristems developed in vitro. The second method is the electroporation of monocot protoplasts with cellulase genes.

The transgene constructs specifically made for plastid transformation are bombarded to monocot multimeristem primordial or alternatively monocot protoplasts are electroporated, and then the transgenic plants are selected. The monocot multimeristem primordial and immature embryo-derived embryogenic cells are bombarded with the chloroplast vectors containing a series of selectable marker genes such as the streptomycin and/or PPT (LIBERTY® herbicide, Bayer CropScience, Research Triangle, N.C.) and/or kanamycin and/or hygromycin resistance genes and the green florescence protein as the reporter gene, and cell lines are selected with resistance to the selective agents. Next, the plants are regenerated the integration of transforming DNA is confirmed. Finally, the plants are produced in contained greenhouses and their pollen grains as well as other cells/tissues are tested for the presence or absence of the transgenes and transgenes products.

Construction of plastid vectors: There are relatively few drugs that can be used for selective amplification of the rare, initial transformed plastid genomes. In this group are streptomycin and spectinomycin, to which resistance is encoded by aadA (Svab, Z., and Maliga, P. (1993). High-frequency plastid transformation in tobacco by selection for a chimeric aadA gene. Proc. Natl. Acad. Sci. USA. 90, 913-917) and kanamycin, to which resistance is encoded by neo (Carrer et al., 1993) and aphA6 (Huang, C. Y., Ayliffe, M. A., and Timmis, J. N. (2003). Direct measurement of the transfer rate of chloroplast DNA into the nucleus. Nature 422, 72-76). Kanamycin has not been the ideal selective agent to select nuclear gene transformants in cereal tissue culture. However, neo and aphA6 also confer resistance to G418 and paromomycin (Toriyama et al., 1988; Torbert et al., 1995), drugs which may be better suited for selection of transplastomic clones in maize. Genes suitable for direct selection of transplastomic clones are termed primary plastid selective markers. There are drugs such as the herbicide phosphinothricin PPT (Lutz, K. A., Knapp, J. E., and Maliga, P. (2001). Expression of bar in the plastid genome confers herbicide resistance. Plant Physiol. 125, 1585-1590) or the antibiotic hygromycin (unpublished) that will confer resistance when most plastid genome copies carry the gene, but are not suitable for the recovery of rare, initial transformation events. These secondary drugs may be more toxic to nontransformed cells than the primary selective agents. To improve the stringency of selection, different primary and secondary selection schemes can be combined. First, the plant is selected for resistance to a primary selective agent, then the primary agent is removed and replaced with a secondary drug. Alternatively, at stage two simultaneous selection for resistance to both drugs can be done. A green fluorescence protein (GFP) marker can further be included in the vectors to facilitate visual verification of transplastomic clones. Accumulation of GFP in chloroplasts can be readily tested by fluorescence microscopy. The selectable markers and the GFP reporter gene are expressed in dicistronic and tricistronic operons.

Alternative selection includes resistance to betaine aldehyde, encoded by the BADH gene (Daniell et al., 2001). Thus far there is no report on recovery of transplastomic clones using vectors in which BADH was the only selective marker. Homologous (maize) and heterologous (non-maize) targeting sequences can be used according to the present invention. In a preferred embodiment homologous maize targeting sequences are used. Selectable marker genes can be used for chloroplast transformation. For example, the eukaryotic protoporphyrineogen oxidase (PPO) herbicide (butafenacil)resistance gene (Lermontova et al., 2000) could be used. The PPO selection interferes with photosynthesis and therefore may work in chloroplast transgenesis. Another selectable marker gene is the phosphomannose-isomerase (PMI; Wright et al., 2001) that enables the recovery of transgenic events in media containing mannose and encourages the growth of transformed tissues rather than killing non-transformed tissues.

Determining selective drug levels, production of multimeristem primordial and immature embryo-derived cell lines, bombardment and regeneration of selected plants: The conditions for selection of transgenic lines by PPT resistance are known in the art. Streptomycin sulfate has been used to select nuclear gene transformants in maize (50 mg/L to 100 mg/L) (Lowe et al., 1995) and transplastomic clones in rice (100 mg/L) (Khan, M. S., and Maliga, P. (1999). Fluorescent antibiotic resistance marker to track plastid transformation in higher plants. Nat. Biotechnol. 17, 910-915). Streptomycin in rice culture inhibits cell division and shoot regeneration. To determine the optimum conditions for selection tissue culture systems, 0, 50, 100, 150 and 200 mg/l of this antibiotic in the monocot embryogenic cell culture or in the multimeristem culture can be used to find the optimum level that could kill non-transgenic cells or multimeristems. Then the optimum level is incorporated into the media after the BIOLISTIC® bombardment to select cells or multimeristems that express the aadA gene encoding streptomycin/spectinomycin resistance. Kill curves can be made for other selectable markers as indicated in this section.

In one embodiment, B73 maize seeds are germinated in Murashige and Skoog (MS) medium for 2 weeks. Shoot apical meristems are dissected under a microscope and cultured in MS medium supplemented with a combination of growth regulators as reported (Zhong H., Srinivasan, C. and Sticklen, M. (1992a). Morphogenesis of corn (Zea mays L.) in vitro II. Transdifferentiation of shoots, tassels, and ear primordial from corn shoot tips. Planta 187: 483-489). Cultures are incubated at room temperature under high intensity fluorescence and incandescent lights for effective photosynthesis for 8 weeks. Multiplied apical shoot meristems (50 and above per clump) are used for plastid bombardment as done for maize nuclear transgenesis (Zhong, H., Warkentin, D., Sun, B., Zhang, S., Wu, T., Wu, R., and Sticklen, M. (1996a). Analysis of the functional activity of the 1.4 kb 5′-region of the rice actin 1 gene in stable transgenic plants of maize (Zea mays L.). Plant Science 116:73-84). Gold particles can be used instead of tungsten as described for embryogenic cell bombardment (Zhong H. and M. Sticklen (1996b). Production of multiple shoots from shoot apical meristems of oat (Avena sativa L.). J. Plant Physiol. 148:667-671) to obtain a better penetration of transgenes in the cells, and in chloroplasts.

In one embodiment, B73 maize seeds can be planted in potting soil in greenhouses and immature embryos are collected as previously described (Armstrong C., Green, C. and Phillips, R. L. (1991). Development and availability of germplasm with high type II culture formation response. Maize Genet. Coop. Newslett. 65: 92-93, Zhang H., Warkentin D., Sun B., Zhong H., and Sticklen M. (1996). The transmission and expression of two transgenes through outcross and self-cross in maize plants. Theor. Appl. Genet. 92: 752-761). Immature embryos are sterilized and cultured in vitro for the production of embryogenic cells as previously described (Zhang H., Warkentin D., Sun B., Zhong H., and Sticklen M. (1996). The transmission and expression of two transgenes through outcross and self-cross in maize plants. Theor. Appl. Genet. 92: 752-761). The above callus line derived from a single embryo of B73 (or Hi II, Monsanto, St. Louis, Mo.) maize showing friable type II embryogenic cells are sieved through 710-μm mesh before bombardment. In this embodiment, these cells are transformed with plastid vectors using the Biolistic® with a helium-powered acceleration machine, PDS 1000 (BioRad, Hercules, Calif.) according to previously described procedures for immature embryo-derived maize cell transformation (Zhang H., Warkentin D., Sun B., Zhong H., and Sticklen M. (1996). The transmission and expression of two transgenes through outcross and self-cross in maize. plants. Theor. Appl. Genet. 92: 752-761). The monocot multimeristem primordial explants and immature embryo-derived embryogenic cells can be bombarded with tungsten or gold particles coated with plastids vector DNAs. The bombarded multimeristem clumps are then further multiplied for 8-10 weeks without any selection as previously described (Zhong H., Srinivasan, C. and Sticklen, M. (1992a). Morphogenesis of corn (Zea mays L.) in vitro II. Transdifferentiation of shoots, tassels, and ear primordial from corn shoot tips. Planta 187: 483-489). A clump of multimeristem (50 or more meristem primordial) can be selected on selective PPT, streptomycin or other selectable media.

Selection of putatively transgenic plants and confirmation of transgene integration and expression: In one embodiment, the multiplied meristems and embryogenic cells can be selected in 5-10 mg/L glufosinate ammonium (PPT) as described previously for nuclearly transformed maize (Zhang H., Warkentin D., Sun B., Zhong H., and Sticklen M. (1996). The transmission and expression of two transgenes through outcross and self-cross in maize plants. Theor. Appl. Genet. 92: 752-761; Zhong, H., Warkentin, D., Sun, B., Zhang, S., Wu, T., Wu, R., and Sticklen, M. (1996a). Analysis of the functional activity of the 1.4 kb 5′-region of the rice actin 1 gene in stable transgenic plants of maize (Zea mays L.). Plant Science 116:73-84; Zhong H. and M. Sticklen (1996b). Production of multiple shoots from shoot apical meristems of oat (Avena sativa L.). J. Plant Physiol. 148:667-671; Zhong H., Teymouri, F., Chapman, B., Maqbool, S., Sabzikar, R., El-Maghraby, Y., Dale, B. and Sticklen, M. (2003). The dicot pea (Posum sativum L.) rbcS transit peptide directs the Alcaligenes eutrophus polyhydroxybutyrate enzymes into the monocot maize (Zea mays L.) chloroplasts. J. Plant Science. Vol. 1-8. In press). In another embodiment, a combination of both PPT and streptomycin, and/or other construct-related selectable chemicals are used to select shoots. The chemically selected multimeristems and shoots are rooted in maize-rooting medium as previously described (Zhong H., Srinivasan, C. and Sticklen, M. (1992a). Morphogenesis of corn (Zea mays L.) in vitro II. Transdifferentiation of shoots, tassels, and ear primordial from corn shoot tips. Planta 187: 483-489) containing the same concentration of selectable chemicals as described therein. Plantlets are potted, acclimated and transferred to greenhouses. As described herein, different primary and secondary selection schemes can be performed. In these embodiments, the plants are selected for resistance to a primary selective agent, then the primary agent is removed and replaced with a secondary drug. In alternative embodiments, at stage two simultaneous selection for resistance to both drugs can be performed.

In one embodiment, the use of the gfp marker gene product in chloroplasts is tested by fluorescence microscopy. This is an easy method to confirm the transgene expression. Polymerase Chain Reaction (PCR) can also be used to confirm the presence of the transgenes. In the PCR method, the DNA is isolated and amplified with primer pairs, one of which is outside the targeting sequence of the transformation vector. The amplified DNA is then electrophoresed to analyzed the products. Southern blot hybridization can be performed to confirm the PCR method as is known by those skilled in the art. The shoots or plantlets which show integration of transgenes in the plastid genome by targeting region and transgene probes, are considered transformed.

Production of R0 plants in contained greenhouses, and testing of pollen grains for the absence of transgenes: In one embodiment, the R0 plant pollen cells are tested by GFP test and through the in situ fluorescence antibody staining and laser microscopy of selectable marker gene products as previously reported (Zhong H., Teymouri, F., Chapman, B., Maqbool, S., Sabzikar, R., El-Maghraby, Y., Dale, B. and Sticklen, M. (2003). The dicot pea (Posum sativum L.) rbcS transit peptide directs the Alcaligenes eutrophus polyhydroxybutyrate enzymes into the monocot maize (Zea mays L.) chloroplasts. J. Plant Science. Vol. 1-8. In press). Then, plants are self and cross pollinated following previous work on maize nuclear transgenesis (Zhong, H., Warkentin, D., Sun, B., Zhang, S., Wu, T., Wu, R., and Sticklen, M. (1996a). Analysis of the functional activity of the 1.4 kb 5′-region of the rice actin 1 gene in stable transgenic plants of maize (Zea mays L.). Plant Science 116:73-84; Zhong H. and M. Sticklen (1996b). Production of multiple shoots from shoot apical meristems of oat (Avena sativa L.). J. Plant Physiol. 148:667-671) and seeds are germinated and the progeny pollens are tested as well. Chloroplast transgenic pollens are then compared with pollens from nuclearly transformed maize plants for the bar gene.

Selection scheme: One difficulty that can be encountered in the development of transplastomic cereals is the initial selection scheme, because after cells/tissues are bombarded about only few plastids may receive the transgenes. Therefore, meristems must be multiplied for about twelve weeks after bombardment before their selection so that more plastids per cell contain the selectable marker gene. For example, bar has proven to be an excellent stable marker gene for transformation of nuclear genomes of cereals, but not for the selection of plastid transformants in rice due to the same reason (Lutz, K. A., Knapp, J. E., and Maliga, P. (2001). Expression of bar in the plastid genome confers herbicide resistance. Plant Physiol. 125, 1585-1590).

On the other hand, spectinomycin (100 mg/L) was the ideal selectable drug in the rice chloroplast transformation studies using pJEK6 (Khan, M. S., and Maliga, P. (1999). Fluorescent antibiotic resistance marker to track plastid transformation in higher plants. Nat. Biotechnol. 17, 910-915). In the drug selection for the present invention, the spectinomycin can be used alone or in combination with PPT. The spectinomycin from the plasmid vector (pJEK6) might be more suitable for the selection of transplastomic maize because based on the design of pJEK6; spectinomycin-resistance is only obtained when the aadA is integrated into the plastid genome. Also, spectinomycin resistance (100 mg/L) was a suitable marker for the initial selection of transplastomic rice clones (Khan, M. S., and Maliga, P. (1999). Fluorescent antibiotic resistance marker to track plastid transformation in higher plants. Nat. Biotechnol. 17, 910-915).

Mixed population of transformed and non-transformed chloroplasts in each cell: It is not impossible to have a mixed population of transformed and non-transformed chloroplasts in a transgenic cell. Although the drug selection by itself should sort and only allow survival of the cells/meristems that carry the selectable transgene in their chloroplasts, the meristems are multiplied for a minimum of 28 weeks (12 weeks without selection and 16 weeks in selection) after the bombardment. Considering that each meristem produces about 50 new meristems every four weeks in our multiplication medium (Zhong H., Srinivasan, C. and Sticklen, M. (1992a). Morphogenesis of corn (Zea mays L.) in vitro II. Transdifferentiation of shoots, tassels, and ear primordial from corn shoot tips. Planta 187: 483-489), there is a chance that all plastids in cells of selected transgenic cells (other than their pollen cells) would carry the transgenes. The seeds of inbred crosses can be multiplied for field testing. This seed multiplication also helps in producing plants which have chloroplasts that are homozygous for the transgenes.

To distinguish whether the cells have such mixed population, immunofluorescent laser microscopy of protoplasts of transgenic plants can be performed to show the presence or absence of the heterologous proteins in the each of chloroplasts. In previous work which shows the localization of three PHB heterologous proteins (nuclear transformation; chloroplast-targeted PHB proteins) in maize chloroplasts, immunofluorescent laser microscopy was performed in this manner(Zhong H., Teymouri, F., Chapman, B., Maqbool, S., Sabzikar, R., El-Maghraby, Y., Dale, B. and Sticklen, M. (2003). The dicot pea (Posum sativum L.) rbcS transit peptide directs the Alcaligenes eutrophus polyhydroxybutyrate enzymes into the monocot maize (Zea mays L.) chloroplasts. J. Plant Science. Vol. 1-8. In press).

Export of transgenes products into the cytoplasm: Presently, not much is known about whether proteins can move out of the chloroplasts and into the cytoplasm. Immunofluorescence laser microscopy of protoplasts of transgenic maize can be performed to identify the localization (and possible movement) of heterologous proteins as was done in previous studies directed to polyhydroxybutyrate localization in chloroplasts of transgenic maize (Zhong H., Teymouri, F., Chapman, B., Maqbool, S., Sabzikar, R., El-Maghraby, Y., Dale, B. and Sticklen, M. (2003). The dicot pea (Posum sativum L.) rbcS transit peptide directs the Alcaligenes eutrophus polyhydroxybutyrate enzymes into the monocot maize (Zea mays L.) chloroplasts. J. Plant Science. Vol. 1-8. In press).

Co-transformation of nuclear and chloroplast genomes: A bombarded gene can integrate in nuclear genome as well as in chloroplast genome. However, aadA gene is under the control of a chloroplast-specific promoter such that spectinomycin-resistance is only obtained when the construct is integrated into the plastid genome because this gene will not express in nuclear genome. Regardless, the simplest method to test which transgenic plants carry the transgenes only in their chloroplasts is to cross breed transgenics (reciprocal breeding) with non-transformed control plants. If chloroplasts contain the transgene and nuclei are free from the transgene, only the crosses in which the transgenic plants are used as the female parent should yield progeny that inherit the transgene.

Plastid genes could rarely hop-over into the nuclear genome, and if they do, they do not function while in nucleus under plastid regulatory sequences. It is well accepted that chloroplasts were prokaryotes that entered plant cells, and there are about 3,000 chloroplast genes in nuclear genome that are believed to have transferred to nuclear genome during the course of evolution (The Arabidopsis Genome Initiative, 2000). The April edition of Nature reports that Jeremy Timmis and his colleagues at the University of Adelaide shown that their injected marker gene in tobacco chloroplasts, could “hop over” into the nucleus about one in 16,000 seedlings (Huang, C. Y., Ayliffe, M. A., and Timmis, J. N. (2003). Direct measurement of the transfer rate of chloroplast DNA into the nucleus. Nature 422, 72-76). This finding has been confirmed recently by Ralph Bock's research group, although the frequency of transfer was lower (Stegemann, S., Hartmann, S., Ruf, S. and Bock, R. (2003) High-frequency gene transfer from the chloroplast genome to the nucleus. Proc. Nat. Acad. Sci).

Chloroplast regulatory sequences (promoters, ribosome binding sites, etc.) do not function in the nucleus. In the experiments on DNA transfer from the plastid genome to the nucleus the neo gene was driven by nuclear regulatory signals, therefore functional in the nucleus. Therefore, should a chloroplast transgene incidentally hop over to nucleus and therefore to pollen grains, such gene would be very unlikely to function in the nucleus (Maliga, P. (2003b). Mobile plastid genes. Nature 422, 31-32). Preferably, the transgenic monocot plant are tested for the presence of transgenes in all plant parts to confirm the lack of transgenes in pollen grains. The absence of pollen gene flow in R1, R2 and in the field (risk assessment studies) and a stable high level production of the transgene product in self and hybrid regenerates are used to confirm the safety of the transgenic monocot plants.

The transgenic monocot plants of the present invention comprise one or more heterologous gene expression cassettes containing DNA encoding at least one fungal or bacterial ligninase, cellulase, or both inserted into the plant's plastid genome. The preferred cellulase is encoded by a DNA from the microorganism Acidothermus cellulolyticus, Thermomonospora fusca, and Trichoderma reesei (Trichoderma longibrachiatum). Other microorganisms which produce cellulases suitable for the present invention include Zymomonas mobilis, Acidothermus cellulolyticus, Cloostridium thermocellum, Eiwinia chrysanthemi, Xanthomonas campestris, Alkalophilic Baccilus sp., Cellulomonas fimi, wheat straw mushroom (Agaricus bisporus), Ruminococcus flavefaciens, Ruminococcus albus, Fibrobacter succinogenes, and Butyrivibrio fibrisolvens.

The preferred ligninase is lignin peroxidase (LIP) encoded in DNA from Phanerochaete chrysosporium or Phlebia radiata. One of the major lignin-degrading, extracellular enzymes produced by P. chrysosporium is LIP. The LIPs are glycosylated heme proteins (MW 38 to 46 kDa) which are dependent on hydrogen peroxide for activity and catalyze the oxidative cleavage of lignin polymer. At least six heme proteins (H1, H2, H6, H7, H8, and H10) with LIP activity have been identified in P. chrysosporium strain BKMF-1767 of which isozymes H2, H6, H8, and H10 are the major LIPs in both static and agitated cultures of P. chrysosporium. However, other fungi which produce ligninases suitable for use in the present invention include Bjerkandera adusta, Trametes hirsuta, Plebia radiata, Pleurotus spp., Stropharia aurantiaca, Hypholoma fasciculare, Trametes versicolor, Gymnopilus penetrnas, Stereum hirsutum, Mycena haematopus, and Armillaria mellea.

In the present invention, the transgenic monocot plant comprises a DNA encoding one or more cellulase proteins wherein the DNA encoding the cellulases are incorporated into the plastid genome of the monocot plant. By sequestering the cellulase proteins in the plant organelle, the cellulase protein is prevented from leaking outside the cytoplasm to harm the plant by degrading the cellulose in the plant's cell wall while the plant is being cultivated.

In a preferred embodiment, the cellulase comprising the cellulase protein is encoded by the EI beta-1,4-endoglucanase precursor gene (e1) of Acidothermus cellulolyticus, the cellobiohydrolase gene (cbh1) of Trichoderma reesei (Trichoderma longibrachiatum), the beta-glucosidase gene from Actinomryces naeslundi, or the glucanhydrolase (dextranase) gene from Streptococcus salivarius. The nucleotide sequence of the e1 DNA is set forth in SEQ ID NO: 1 (GenBank Accession No. U33212), which encodes the cellulase with the amino acid sequence set forth in SEQ ID NO: 7. SEQ ID NO: 4 provides the nucleotide sequence of the beta-glucosidase gene from Actinomyces naeslundi (GenBank Accession No. AY029505), which encodes the beta-glucosidase with the amino acid sequence set forth in SEQ ID NO: 8. SEQ ID NO: 3 provides the nucleotide sequence of the dextranase gene from Streptococcus salivarius (GenBank Accession No. D29644), which encodes a glucanhydrolase with the amino acid sequence set forth in SEQ ID NO: 9. The nucleotide sequence of cbh1 is set forth in SEQ ID NO: 2 (GenBank Accession No. E00389), which encodes the cellulase that includes the joined exons from positions 210 to 261, 738 to 1434, and 1498-1881.

In a preferred embodiment of the invention, the ligninase comprising the ligninase protein is encoded by the lignin peroxidase gene (LIP) genes ckg4 (H2) and ckg5 (H10) of Phanerochaete crysosporium (de Boer et al, Gene 6: 93-102 (1987), Corrigendum in Gene 69: 369 (1988)). The nucleotide sequence of the ckg4 gene is set forth in SEQ ID NO: 5(GenBank Accession No. M18743), which encodes the amino acid with the sequence set forth in SEQ ID NO: 10. The nucleotide sequence of the ckg5 gene is set forth in SEQ ID NO: 6(GenBank Accession No. M18794), which encodes the amino acid with the sequence set forth in SEQ ID NO: 11.

The heterologous gene expression cassettes can be constructed using conventional molecular biology cloning methods. In a particularly convenient method, PCR is used to produce the nucleotide fragments for constructing the gene expression cassettes. By using the appropriate PCR primers, the precise nucleotide regions of the above DNAs can be amplified to produce nucleotide fragments for cloning. By further including in the PCR primers restriction enzyme cleavage sites which are most convenient for assembling the heterogeneous gene expression cassettes (e.g., restriction enzyme sites that are not in the nucleotide fragments to be cloned), the amplified nucleotide fragments are flanked with the convenient restriction enzyme cleavage sites for assembling the nucleotide fragments into heterogeneous gene expression cassettes. The amplified nucleotide fragments are assembled into the heterogeneous gene expression cassettes using conventional molecular biology methods. Based upon the nucleotide sequences provided herein, how to construct the heterogeneous gene expression cassettes using conventional molecular biology methods with or without PCR would be readily apparent to one skilled in the art.

In a further embodiment of the present invention, the transgenic plant comprises more than one heterogeneous gene expression cassette. For example, the transgenic plant comprises a first cassette which contains a DNA encoding a ligninase protein, and one or more cassettes each containing a DNA encoding a particular cellulase protein.

In a further still embodiment, the transgenic plant comprises DNA encoding the ligninase protein such as the ckg4 or ckg5 LIP, an endoglucanase protein such as the e1 protein, and a cellobiohydrolase protein such as the cbh1 protein. In a further still embodiment, the transgenic plant comprises DNA encoding the ligninase protein such as the ckg4 or ckg5 LIP, an endoglucanase protein such as the e1 protein, a cellobiohydrolase protein such as the cbh1 protein, a beta-glucosidase, and a glucanhydrolase.

To make the transgenic monocot plants of the present invention, plant material such as meristem primordial tissue is transformed with plasmids, each containing a particular heterogeneous gene expression cassette using the Biolistic bombardment method as described in Example 5 and in U.S. Pat. No. 5,767,368 to Zhong et al. Further examples of the Biolistic bombardment method are disclosed in U. S. application Ser. No. 08/036,056 and U.S. Pat. No. 5,736,369 to Bowen et al. Each heterogeneous gene expression cassette is separately introduced into a plant tissue and the transformed tissue propagated to produce a transgenic plant that contains the particular heterogeneous gene expression cassette. Thus, the result is a transgenic plant containing the heterogeneous gene expression cassette expressing a ligninase such as ckg4 or ckg5, a transgenic plant containing a heterogeneous gene expression cassette expressing endoglucanase such as e1, a transgenic plant containing a heterogeneous gene expression cassette expressing a cellobiohydrolase such as cbh1, a transgenic plant containing a heterogeneous gene expression cassette expressing an exoglucanase such as beta-glucosidase, and a transgenic plant containing a heterogeneous gene expression cassette expressing an exoglucanase such as glucanhydrolase.

Alternatively, transformation of corn plants can be achieved using electroporation or bacterial mediated transformation using a bacterium such as Agrobacterium tumefaciens to mediate the transformation of corn root tissues (see Valvekens et al. Proc. Nat'l. Acad. Sci. USA. 85: 5536-5540 (1988)) or meristem primordia.

In a preferred embodiment of the present invention, the transgenic plant comprises one or more ligninase proteins and one or more cellulase proteins. Construction of the preferred transgenic plant comprises making first generation transgenic monocot plants as above, each comprising a ligninase protein, and transgenic monocot plants as above, each comprising a cellulase protein. After each first generation transgenic plant has been constructed, progeny from each of the first generation transgenic monocot plants are cross-bred by sexual fertilization to produce second generation transgenic monocot plants comprising various combinations of both the ligninase protein and the cellulase protein.

For example, various combinations of progeny from the first generation transgenic monocot plants are cross-bred to produce second generation transgenic monocot plants that contain ckg4 and cbh1, e1, beta-glucosidase, or ckg5; second generation transgenic monocot plants that contain ckg5 and cbh1, e1, or beta-glucosidase; second generation transgenic monocot plants that contain e1 or beta glucosidase, and a second generation transgenic plant that contains e1 and beta-glucosidase.

Progeny of the second generation transgenic monocot plants are cross-bred by sexual fertilization among themselves or with first generation transgenic monocot plants to produce third generation transgenic monocot plants that contain one or more ligninases, one or more cellulases, or combinations thereof.

For example, cross-breeding a second generation transgenic plant containing ckg4 and cbh1 with a second generation transgenic plant containing e1 and beta-glucosidase produces a third generation transgenic plant containing ckg4, cbh1, e1, and beta-glucosidase. The third generation transgenic plant can be cross-bred with a first generation transgenic plant containing ckg5 to produce a fourth generation transgenic plant containing ckg4, ckg5, cbh1, e1, and beta-glucosidase.

Other transgenic monocot plants with various combinations of ligninases and cellulases can be made by cross-breeding progeny from particular transgenic monocot plants. Zhang et al, Theor. Appl. Genet. 92: 752-761, (1996), Zhong et al, Plant Physiol. 110: 1097-1107, (1996);, and Zhong et al, Planta, 187: 483-489, (1992) provide methods for making transgenic monocot plants by sexual fertilization.

Alternatively, plant material is transformed as above with a plasmid containing a heterologous gene expression cassette encoding the ligninase protein. The transgenic plant is recovered from the progeny of the transformed plant material. Next, plant material from the transgenic plant is transformed with a second plasmid containing a heterologous gene expression cassette encoding the cellulase protein and a second selectable marker. The transgenic plant is recovered from the progeny of the transformed plant material. Transgenic monocot plants containing any combination of ligninases and cellulases can be made by the above method.

In a preferred embodiment, the above heterologous gene expression cassettes further include therein nucleotide sequences that encode one or more selectable markers which enable selection and identification of transgenic monocot plants that express the modified cellulase of the present invention. Preferably, the selectable markers confers additional benefits to the transgenic plant such as herbicide resistance, insect resistance, and/or resistance to environmental stress.

Alternatively, the above transformations are performed by co-transforming the plant material with a first plasmid containing a heterologous gene expression cassette encoding a selectable marker and a second plasmid containing a heterologous gene expression cassette encoding a ligninase or cellulase protein. The advantage of using a separate plasmid is that after transformation, the selectable marker can be removed from the transgenic plant by segregation, which enables the selection method for recovering the transgenic plant to be used for recovering transgenic monocot plants in subsequent transformations with the first transgenic plant.

Examples of markers that provide resistance to herbicides include, but are not limited to, the bar gene from Streptomyces hygroscopicus encoding phosphinothricin acetylase (PAT), which confers resistance to the herbicide glufonsinate; mutant genes which encode resistance to imidazalinone or sulfonylurea such as genes encoding mutant form of the ALS and AHAS enzyme as described by Lee at al. EMBO J. 7: 1241 (1988) and Miki et al, Theor. Appl. Genet. 80: 449 (1990), respectively, and in U.S. Pat. No. 5,773,702 to Penner et al.; genes which confer resistance to glycophosphate such as mutant forms of EPSP synthase and aroA; resistance to L-phosphinothricin such as the glutamine synthetase genes; resistance to glufosinate such as the phosphinothricin acetyl transferase (PAT and bar) gene; and resistance to phenoxy proprionic acids and cycloshexones such as the ACCase inhibitor-encoding genes (Marshall et al. Theor. Appl. Genet. 83: 435 (1992)). The above list of genes which can import resistance to an herbicide is not inclusive and other genes not enumerated herein but which have the same effect as those above are within the scope of the present invention.

Examples of preferred genes which confer resistance to pests or disease include, but are not limited to, genes encoding a Bacillus thuringiensis protein such as the delta-endotoxin, which is disclosed in U.S. Pat. No. 6,100,456 to Sticklen et al.; genes encoding lectins, (Van Damme et al, Plant Mol. Biol. 24: 825 (1994)); genes encoding vitamin-binding proteins such as avidin and avidin homologs which can be used as larvicides against insect pests; genes encoding protease or amylase inhibitors, such as the rice cysteine proteinase inhibitor (Abe et al, J. Biol. Chem. 262: 16793(1987)) and the tobacco proteinase inhibitor I (Hubb et al, Plant Mol. Biol. 21: 985(1993)); genes encoding insect-specific hormones or pheromones such as ecdysteroid and juvenile hormone, and variants thereof, mimetics based thereon, or an antagonists or agonists thereof; genes encoding insect-specific peptides or neuropeptides which, upon expression, disrupts the physiology of the pest; genes encoding insect-specific venom such as that produced by a wasp, snake, etc.; genes encoding enzymes responsible for the accumulation of monoterpenes, sesquiterpenes, asteroid, hydroxaminc acid, phenylpropanoid derivative or other non-protein molecule with insecticidal activity; genes encoding enzymes involved in the modification of a biologically active molecule (see U.S. Pat. No. 5,539,095 to Sticklen et al., which discloses a chitinase that functions as an anti-fungal); genes encoding peptides which stimulate signal transduction; genes encoding hydrophobic moment peptides such as derivatives of Tachyplesin which inhibit fungal pathogens; genes encoding a membrane permease, a channel former or channel blocker (for example cecropin-beta lytic peptide analog renders transgenic tobacco resistant to Pseudomonas solanacerum) (Jaynes et al. Plant Sci. 89: 43 (1993)); genes encoding a viral invasive protein or complex toxin derived therefrom (viral accumulation of viral coat proteins in transformed cells of some transgenic cereal plants impart resistance to infection by the virus the coat protein was derived as shown by Beachy et al. Ann. Rev. Phytopathol. 28: 451 (1990); genes encoding an insect-specific antibody or antitoxin or a virus-specific antibody (Tavladoraki et al. Nature 366: 469(1993)); and genes encoding a developmental-arrestive protein produced by a plant, pathogen or parasite which prevents disease. The above list of genes which can import resistance to disease or pests is not inclusive and other genes not enumerated herein but which have the same effect as those above are within the scope of the present invention.

Examples of genes which confer resistance to environmental stress include, but are not limited to, mtld and HVA1, which are genes that confer resistance to environmental stress factors; rd29A and rd19B, which are genes of Arabidopsis thaliana that encode hydrophilic proteins which are induced in response to dehydration, low temperature, salt stress, or exposure to abscisic acid and enable the plant to tolerate the stress (Yamaguchi-Shinozaki et al, Plant Cell 6: 251-264 (1994)). Other genes contemplated can be found in U.S. Pat. Nos. 5,296,462 and 5,356,816 to Thomashow. The above list of genes, which can import resistance to environmental stress, is not inclusive and other genes not enumerated herein but which have the same effect as those above are within the scope of the present invention.

Thus, it is within the scope of the present invention to provide transgenic monocot plants which express one or more ligninase proteins, one or more cellulase proteins, and one or more of any combination of genes which confer resistance to an herbicide, pest, or environmental stress.

To degrade the lignocellulose in the leaves and stalks of the transgenic monocot plants of the present invention, the transgenic plant is ground up to produce a plant material using methods currently available in the art to disrupt a sufficient number of the plant organelles containing the ligninase and cellulase therein. The ligninase and cellulase degrade the lignocellulose of the transgenic plant into fermentable sugars, primarily glucose, and residual solids. The fermentable sugars are used to produce ethanol or other products.

The transgenic monocot plants can be processed to ethanol in an improvement on the separate saccharification and fermentation (SHF) method (Wilke et al., Biotechnol. Bioengin. 6: 155-175 (1976)) or the simultaneous saccharification and fermentation (SSF) method disclosed in U.S. Pat. No. 3,990,944 to Gauss et al. and U.S. Pat. No. 3,990,945 to Huff et al. The SHF and SSF methods require pre-treatment of the plant material feedstock with dilute acid to make the cellulose more accessible followed by enzymatic hydrolysis using exogenous cellulases to produce glucose from the cellulose, which is then fermented by yeast to ethanol. In some variations of the SHF or SSF methods, the plant material is pre-treated with heat or with both heat and dilute acid to make the cellulose more accessible.

An SHF or SSF method that uses the transgenic plant material of the present invention as the feedstock is an improvement over the SHF or SSF method because the transgenic plant material contains its own cellulases and ligninases or cellulases. Therefore, exogenous ligninases and/or cellulases do not need to be added to the feedstock. Furthermore, because particular embodiments of the transgenic plant material produce ligninase, the need for pre-treatment of the plant material in those embodiments before enzymatic degradation is not necessary. In a further improvement over the SHF method, the transgenic plant material is mixed with non-transgenic plant material and the mixture processed to ethanol.

The maize transgenic technology of the present invention eliminates or dramatically reduces transgene transfer through pollen flow. The following steps are taken to achieve this objective:

1. Develop spectinomycin selective drug kill curve, bombard maize multimeristem primordial with the chloroplast vector pJEK6 containing the spectinomycin and PPT (LIBERTY® herbicide, Bayer CropScience, Research Triangle, N.C.) resistance genes, and select maize meristem lines with resistance to the selective agents.

2. Regenerate plants and confirm the integration of the chloroplast DNA-targeted genes in chloroplasts.

3. Produce reciprocal crosses between transgenic and non-transgenic plants in greenhouses, and confirm whether chloroplast transgenes are exclusively inherited through the egg cells only.

Production of multimeristem primordial, development of kill curve for spectinomycin resistance, and bombardment:

a. Shoot apical meristem multiplication: B73 maize inbred line seeds are germinated in Murashige and Skoog (MS) medium (Murashige T. and Skoog F. (1972). A revised medium for rapid growth and bioassay with tobacco tissue culture. Plant Physiol. 15; 473-497) for two weeks. Shoot apical meristems are dissected under a microscope and cultured in MS medium supplemented with a combination of growth regulators as reported (Zhong H., Srinivasan, C. and Sticklen, M. (1992a). Morphogenesis of corn (Zea mays L.) in vitro II. Transdifferentiation of shoots, tassels, and ear primordial from corn shoot tips. Planta 187: 483-489). Cultures are incubated at room temperature under high intensity fluorescence and incandescent lights for effective photosynthesis for eight weeks.

b. Development of spectinomycin tolerance kill curve: Based on literature on transformation of cereals, all selectable drugs that have worked on transgenic rice have also worked well in transgenic maize, and the fact that spectinomycin has shown to be a good selectable drug for chloroplast-transgenic rice is most promising. To develop a kill curve on level of spectinomycin tolerance of maize multimeristems, multiple apical meristems (50 and above per clump) are used for the development of the spectinomycin tolerance kill curve. Kill curve are developed to determine the optimum level of spectinomycin that can kill the untransformed maize multimeristems. Then this level is used in selecting our bombarded meristems. Khan and Maliga (1999) used a total of 100 mg/L spectinomycin to select chloroplast-transgenic rice cell lines using the same construct (i.e. pJEK6). Therefore to develop a kill curve for maize shoot apical multimeristems, we will use 0, 50, 100, 150 and 200 mg/l spectinomycin in our multiplication medium to find the optimum level that could kill non-transgenic cells in the meristem tissues. There are ten Petri dishes in each concentration, each dish containing four multimeristem clumps of about 50 meristems. The experiment is repeated three times in space under our tissue culture laboratory conditions. Data is statistically analyzed using the completely randomized block design via SAS.

c. Biolistic® bombardment: Multiplied apical shoot meristem clumps are used for bombardment of the chloroplast-specific DNA using our Biolistic® device, as we did for maize nuclear transgenesis (Zhong, H., Warkentin, D., Sun, B., Zhang, S., Wu, T., Wu, R., and Sticklen, M. (1996a). Analysis of the functional activity of the 1.4 kb 5′-region of the rice actin 1 gene in stable transgenic plants of maize (Zea mays L.). Plant Science 116:73-84; Zhong H. and M. Sticklen (1996b). Production of multiple shoots from shoot apical meristems of oat (Avena sativa L.). J. Plant Physiol. 148:667-671; Zhong H., Teymouri, F., Chapman, B., Maqbool, S., Sabzikar, R., El-Maghraby, Y., Dale, B. and Sticklen, M. (2003). The dicot pea (Posum sativum L.) rbcS transit peptide directs the Alcaligenes eutrophus polyhydroxybutyrate enzymes into the monocot maize (Zea mays L.) chloroplasts. J. Plant Science. Vol. 1-8. In press). Gold particles are used instead of tungsten as we did to obtain a better penetration of transgenes in the cells nuclei, and here in the chloroplasts.

More multimeristem multiplication after bombardment: The drug such as the herbicide phosphinothricin or PPT (Lutz, K. A., Knapp, J. E., and Maliga, P. (2001). Expression of bar in the plastid genome confers herbicide resistance. Plant Physiol. 125, 1585-1590) will confer resistance when most of the chloroplast genome copies carry the bar gene (communication with Dr. Maliga). Therefore, the bombarded meristems are multiplied for at least twelve more weeks after the bombardment until many of the chloroplasts in each cell contain the selectable marker bar transgene. In this step, the bombarded multiple meristem clumps are divided and subcultured (two-week interval) for another twelve weeks in our maize apical meristem multiplication medium to assure that chloroplasts/cells that have received the transgenes have multiplied sufficiently in the multimeristem clumps.

Selection of putatively transgenic plants: Although the simplest, this is probably the most critical part of the process because it is important not to keep escapes (cells which do not carry transgenes in their chloroplasts) nor kill all meristem cells during drug tolerance selections.

As indicated above, the aadA gene in pJEK6 is under the control of a chloroplast promoter, and spectinomycin-resistance is only obtained when the construct is integrated into the plastid genome. Therefore, the selection scheme must definitely include medium containing an optimal level of spectinomycin to which resistance is encoded by aadA (Scab and Maliga, 1993).

Also, selection to PPT is beneficial because bar is an excellent marker gene for selecting transgenic maize multimeristems (Zhong H., Teymouri, F., Chapman, B., Maqbool, S., Sabzikar, R., El-Maghraby, Y., Dale, B. and Sticklen, M. (2003). The dicot pea (Posum sativum L.) rbcS transit peptide directs the Alcaligenes eutrophus polyhydroxybutyrate enzymes into the monocot maize (Zea mays L.) chloroplasts. J. Plant Science. Vol. 1-8. In press; Zhong, H., Warkentin, D., Sun, B., Zhang, S., Wu, T., Wu, R., and Sticklen, M. (1996a). Analysis of the functional activity of the 1.4 kb 5′-region of the rice actin 1 gene in stable transgenic plants of maize (Zea mays L.). Plant Science 116:73-84, Zhong H. and M. Sticklen (1996b). Production of multiple shoots from shoot apical meristems of oat (Avena sativa L.). J. Plant Physiol. 148:667-671).

Spectinomycin and PPT selectable drugs can be both used individually and in combination on the pJEK6-bombarded multiplied meristems. To do so, 250 Petri dishes were chosen each containing four bombarded (and further multiplied) apical meristem clumps (about 50 meristems per clump). A selection experiment is designed with three groups and two phases. Phase I is kept under eight weeks of selection pressure. Phase II is a continuation of Phase I and is kept under further selection pressure for another eight weeks. There are 100 Petri dishes in Group I, 100 Petri dishes in Group II and 50 Petri dishes in Group III of this experiment (see Table 1 below). Each Petri dish contains four multiple shoot meristem clumps, each clump with about 50 meristem.

TABLE 1
Summary of Vigorous Selection Schemes for
Chloroplast-Transgenic Shoot Apical Meristems

| | First Stage | Second Stage |
|---|---|---|
| | (8 weeks) | (another 8 weeks) |
| Group I | Selection A: Petri | Selection A₁: Keep |
| | dishes contain the | surviving clumps of |
| | multiplication | 50 dishes to A |
| | medium + the optimum | Selection A_{2:} |
| | concentration of | Transfer surviving |
| | spectinomycin | clumps of 50 dishes |
| | | to B |
| Group | Selection B: Petri | Selection B₁: Keep |
| II | dishes contain the | surviving clumps of |
| | multiplication | 50 dishes to B |
| | medium + 10 mg/L PPT | Selection B_{2:} |
| | | Transfer surviving |
| | | clumps of 50 dishes |
| | | to A. |
| Group | Selection C: Petri | Selection C_{1:} |
| III | dishes contain the | Keep all surviving |
| | multiplication | clumps in C |
| | medium + the optimum | |
| | concentration of | |
| | spectinomycin + 10 mg/L | |
| | PPT | |

As Table 1 above shows, the bombarded cultures are selected under spectinomycin (Selection A), PPT (Selection B) or combination of both (Selection C). After eight weeks of selection pressure, surviving clumps of half of the Selection A Petri dishes remain in Selection A for another eight weeks (Selection A₁), and the surviving clumps of the other half of Selection A Petri dishes are transferred to Selection B for another eight weeks. The same steps are applied to the Selection B maintenance. The surviving clumps of all Petri dishes of Selection C are kept under the same selection pressure for another eight weeks.

All surviving clumps are transferred to their own fresh media every two weeks to assure that depletion of media ingredient will not affect the cultures. In the Second Stage, if clumps are very large (more than 50 meristem per clump), each clump is divided prior to their transfer to A₁, A₂, B₁, B₂, or C₁. At the end of 16 weeks of selection pressure, data is collected from A₁, A₂, B₁, B₂, and C₁. The above experiment is repeated twice. Statistical analysis (completely randomized block design) is performed using SAS.

All surviving multiple shoot clumps are used in our molecular analysis to confirm the integration and expression of transgenes. However, the most desirable selection scheme can be chosen based on the statistical analysis. All survived meristems are regenerated into shoots in vitro. Leaf samples are harvested for chloroplast isolation and Polymerase Chain Reaction (Mullis K and F. Faloona (1987). Specific synthesis of DNA in vitro via a polymerase chain reaction. Methods in Enzymology. 155:335-350) analysis of chloroplast DNA. Because of the large number of the selected meristems, PCR is used to screen ‘putatively transgenic meristems.’ PCR positive shoots are rooted and plantlets are transferred to soil, acclimated and grown in greenhouses for further molecular testing.

If meristems do not survive any of the above selection schemes, the number of weeks of no-selection subcultures (D2 above) can be increased and selection repeated as explained previously. If no meristem survives in 10 mg/L PPT, a new kill curve can also be designed and implemented for PPT selection as our knowledge of 10 mg/L PPT selection has been for unclearly transformed maize with bar. However, it would not be expected that any different PPT concentration is necessary for selecting apical shoot clumps that have integrated and expressed the bar in their chloroplasts.

PCR analysis, isolation of chloroplasts and confirmation of the integration and transcription of transgenes: When shootlets are small, PCR can be used to screen for ‘putatively transformed meristem.’ Only plantlets that are shown to have an insert in the chloroplast DNA are propagated further as putative transformants. One can also perform locus-specific PCR, followed by restriction or sequencing. However, the shootlets can simply be screened for putative transformants before performing Southern blots (Southern E. M. (1975). Detection of specific sequences among DNA fragments extracted by gel electrophoresis. J. Mol. Biol. 98: 508-517) of chloroplast DNA to screen among hundreds of selected meristems.

To confirm the integration of genes in plant chloroplasts, chloroplast DNA is isolated from greenhouse-grown putatively transgenic and control (untransformed) plants following previously described procedures (Zhong H., Teymouri, F., Chapman, B., Maqbool, S., Sabzikar, R., El-Maghraby, Y., Dale, B. and Sticklen, M. (2003). The dicot pea (Posum sativum L.) rbcS transit peptide directs the Alcaligenes eutrophus polyhydroxybutyrate enzymes into the monocot maize (Zea mays L.) chloroplasts. J. Plant Science. Vol. 1-8. In press). Southern blot analysis of chloroplast DNA is performed as described (Zhong H., Teymouri, F., Chapman, B., Maqbool, S., Sabzikar, R., El-Maghraby, Y., Dale, B. and Sticklen, M. (2003). The dicot pea (Posum sativum L.) rbcS transit peptide directs the Alcaligenes eutrophus polyhydroxybutyrate enzymes into the monocot maize (Zea mays L.) chloroplasts. J. Plant Science. Vol. 1-8. In press). The transgene copy number can also be calculated using Southern blots and/or via SYBR Green I dye Real-Time PCR method as done for transgenic rice (Ahmad A., Zhong, H., Wang, W. and Sticklen, M. (2002). Shoot apical meristem: In vitro regeneration and morphogenesis in wheat (Triticum aestivum L.). In-Vitro Cellular & Developmental Biology (IVCDB)-PLANT. 38: 163-167). To confirm the expression of genes at the transcription level, RNA is isolated from chloroplasts as described (Sears, B. B. and R. G. Herrman (1985). Plastome mutation is a post-transcriptional defect. Current Genetics 9:521-528) to be used in maize Northern analysis (Zhong, H., Warkentin, D., Sun, B., Zhang, S., Wu, T., Wu, R., and Sticklen, M. (1996a). Analysis of the functional activity of the 1.4 kb 5′-region of the rice actin 1 gene in stable transgenic plants of maize (Zea mays L.). Plant Science 116:73-84; Zhong H. and M. Sticklen (1996b). Production of multiple shoots from shoot apical meristems of oat (Avena sativa L.). J. Plant Physiol. 148:667-671).

Reciprocal cross breeding and testing of the progeny: The transgenic maize can be cross bred with non-transgenic B73 maize pollen in the greenhouse, as described previously (Zhong H. and M. Sticklen (1996b). Production of multiple shoots from shoot apical meristems of oat (Avena sativa L.). J. Plant Physiol. 148:667-671). Seeds from those crosses can be germinated and the progeny tested for the presence or absence of transgenes. If successful, only the crosses in which the transgenic plants are used as the female parent will yield progeny that inherit the transgene. Otherwise, there is a possibility that the transgene has incorporated in nuclear genome.

### EXAMPLE

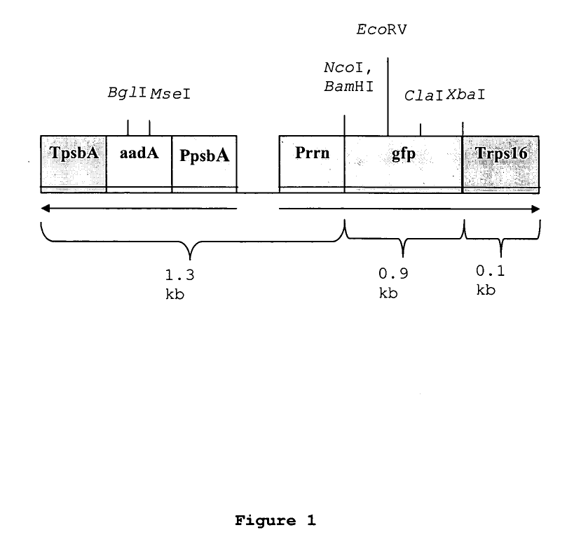

A chloroplast-specific plasmid construct was used, which was obtained from Dr. Maureen Hanson of Cornell University, as illustrated in FIG. 1. The construct is transferred to maize. The illustrated region is flanked by trnv-rps 12/7 plastid derived sequences. aadA represents the gene encoding aminoglycoside adenyl transferase, which renders resistance to spectinomycin and streptomycin. gfp is the green fluorescent protein gene from jellyfish. TpsbA represents the 3′ untranslated region of the psbA gene. PpsbA represents the regulatory region of the psbA gene. Prrn represents the regulatory region of the prrn promoter. Trps16 represents the 3′ region of the ribosomal protein gene. BglI, MseI, EcoRV, NcoI, BamHHI, ClaI and XbaI identify the corresponding restriction endonuclease recognition sites. Insert sizes are designated in kilobases (kB).

Explant Development for Biolistic bombardment: Development of multiple shoot meristems was performed as described in U.S. Pat. No. 5,767,368 to Zhong et al. Briefly, mature seeds of HNP were surface sterilized with 70% ethanol and 2.6% sodium hypochlorite and germinated onto MS medium without hormones. A one-cm-long section of a 7-d-old seedling containing a shoot tip, leaf primordial, and a portion of young leaves and stem proximal to the shoot meristems were excised and cultured in darkness for 2 weeks, and then maintained in light at 4-week intervals onto MS medium, supplemented with 500 mg/l casein hydrolysate, 2 mg/l BAP, 0.5 mg/l 2, 4-D, 3.0 gm/l gelrite and 30 gm/l sucrose or 30 gm/l maltose at pH 5.8.

Development of immature embryo-derived callus for bombardment: Hi II corn germplasm was used for the initiation of callus. Immature embryos were placed on N6-based media, supplemented with 25 mM proline, 100 mg/l casein hydrolysate, 50 μM silver nitrate, 2 mg/l 2, 4-D, 30 gm/l sucrose and 0.25% gelrite at 28° C. in the dark. Callus produced from the scutellum was selected and subcultured every two weeks to same medium until bombardment experiments conducted.

Determine selective drugs: The construct as illustrated in FIG. 1 contains the aadA selectable marker gene. The aadA is considered resistant to spectinomycin and streptomycin. A kill curve was made using immature embryo-derived calli and multimeristems with 0.0, 750, 1,500 and 3,000 mg/L of spectinomycin and 0.0, 200, 400, 600 and 800 mg/L of streptomycin. The pLAAgfp-bombarded calli and multimeristems were transferred after they were multiplied for 4 weeks to the above concentrations to determine the optimum level of antibiotic that can select transformants. Up to 1,500 mg/L of spectinomycin and up to 200 mg/L of streptomycin were not sufficient to kill maize cells.

The maize multimeristem primordial and/or immature embryo-derived embryogenic cells were bombarded with the chloroplast vectors. The pLAAgfp was bombarded into immature embryo-derived cell cultures and into multiple shoot apical meristems as shown in Table 2 and Table 3 below.

TABLE 2
Shoot multimeristems Bombarded with pLAAgfp

| | | R. disk | Dis- | | |
|---|---|---|---|---|---|
| Date | No. of times | pressure | tance | μ | |
| bombarded | bombarded | (p.s.i) | (cm) | carrier | Present status |
| Jan. 31, 2005 | 4 | 1550 | 6.5 | Gold | On |
| | | | | | multiplication |
| Feb. 8, 2005 | 4 | 1550 | 6.5 | Gold | On |
| | | | | | multiplication |
| Feb. 23, 2005 | 4 | 1550 | 6.5 | Gold | On |
| | | | | | multiplication |

TABLE 3
Immature-Embryo-Derived Cells Bombarded with pLAAgfp.

| | | Pres- | Dis- | | |
|---|---|---|---|---|---|
| Date | No. of times | sure | tance | μ | |
| bombarded | bombarded | (p.s.i) | (cm) | carrier | Present status |
| | 1 | 1100 | 3.5 | Gold | On proliferation |
| Jan. 19, 2005 | 2 | 1100 | 6.5 | Gold | *On proliferation |
| | | | | | & selection |
| | 1 | 1100 | 12.5 | Gold | On proliferation |
| | 1 | 1100 | 9.5 | Gold | On proliferation |
| | 2 | 1100 | 6.5 | Gold | On proliferation |
| Jan. 28, 2005 | 1 | 1100 | 12.5 | Gold | On proliferation |
| | 1 | 1100 | 3.5 | Gold | On proliferation |
| | 1 | 1100 | 6.5 | Gold | On proliferation |
| | 1 | 1100 | 3.5 | Gold | On proliferation |
| Feb. 17, 2005 | 2 | 1100 | 9.5 | Gold | On proliferation |
| | 1 | 1100 | 12.5 | Gold | On proliferation |
| Feb. 23, 2005 | 2 | 1100 | 9.5 | Gold | On proliferation |
| | 2 | 1100 | 9.5 | Gold | On proliferation |

Results: The GFP expression was observed under a UV microscope in the experiment marked with asterisk (*) in Table 3. The expression of GFP in the maize cells was observed at four weeks after being bombarded with pLAAgfp.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the Claims attached herein.

1. A method of genetically engineering a monocot plant so that one or more transgenes stably integrate within plastid genomic DNA of the monocot plant comprising:
(a) providing a monocot plant and a targeting construct comprising one or more transgenic sequences;
(b) introducing the targeting DNA sequence into the monocot plant by means of transformation of multimeristems;
(c) selecting transformed monocot plant cells; and
(d) growing the transformed monocot plant, so that the transformed monocot plant has one or more transgenes stably integrated within the plastid genomic DNA.

2. The method of claim 1 wherein targeting construct further comprises a first targeting sequence complimentary to a first plastid genomic DNA sequence of a monocot plastid, a second targeting sequence complimentary to a second plastid genomic DNA sequence of a monocot plastid, a plastid specific promoter linked between the first targeting sequence and the second targeting sequence, wherein the one or more transgenic DNA sequences are between the first targeting sequence and the second targeting sequence and are operationally linked to the plastid specific promoter segment.

3. The method of claim 1 wherein the transformed monocot plant cells are selected by means of herbicide resistance or antibiotic resistance.

4. The method of claim 1 wherein the one or more transgenic DNA sequences encode for at least one enzyme.

5. The method of claim 1 wherein the monocot plant is selected from the group consisting of maize, sugar cane, switchgrass and other perennial grasses.

6. The method of claim 1 wherein the plastid is protoplastid or a chloroplast.

7. The method of claim 1 wherein the one or more transgenic DNA sequences confer herbicide resistance, insect resistance, or environmental stress resistance.

8. The method of claim 1 wherein the targeting construct further comprises a DNA encoding a selectable marker operably linked to a constitutive promoter.

9. The method of claim 8 wherein the DNA encoding the selectable marker provides the transgenic plant with resistance to an antibiotic, an herbicide, or to environmental stress.

10. The method of claim 9 wherein the DNA encoding resistance to the herbicide is a DNA encoding phosphinothricin acetyl transferase which confers resistance to the herbicide phosphinothricin.

11. The method of claim 8 wherein the DNA encoding the selectable marker provides the transgenic plant with resistance to an antibiotic selected from the group consisting of hygromycin, streptomycin, spectinomycin, G418, and paromomycin.

12. The method of claim 2 wherein the plastid specific promoter is selected from the group consisting of Prrn, PrpoB, PrbcL, PpsbA and PpsbD.
